(19) United States
(12) Patent Application Publication
(10) Pub. No.: US 2014/0333441 A1
(43) Pub. Date: Nov. 13, 2014

(54) Diaper Monitor

(71) Applicants: Anthony Solazzo, Watchung, NJ (US); Herbert Heflich, Martinsville, NJ (US); Michael Vaillancourt, Chester, NJ (US)

(72) Inventors: Anthony Solazzo, Watchung, NJ (US); Herbert Heflich, Martinsville, NJ (US); Michael Vaillancourt, Chester, NJ (US)

(21) Appl. No.: 13/889,829

(22) Filed: May 8, 2013

(51) Int. Cl. A61F 13/42

(52) U.S. Cl. CPC A61F 13/42; A61F 2013/424
USPC 340/573.5; 604/361

(57) ABSTRACT

The diaper monitor has a sensor housing that can be slipped into a pocket formed in a diaper at the time of use and secured in place by a pivotal clip. The housing has a pair of conductive strips for sensing wetting of a diaper and a responsive wireless transmitter to emit a responsive signal. The clip may also complete a circuit in the wireless transmitter when moved into the closed position.

This invention relates to a diaper monitor. More particularly, this invention relates to a reusable diaper monitor.

As is known, various techniques have been proposed for monitoring the wetness of diapers, such as infant diaper, incontinence diapers and seniors' diapers. Such techniques are described in published US Patent Application 2012/0206265.

Also described in published US Patent Application 2012/0206265 is a disposable diaper with a wireless alarm system involving a single diaper and a single frequency transmission to set off an alarm when the diaper is wet. The disposable diaper is disclosed as containing a conductive strip with two separated conductive bands so that when conductive liquid (urine) contacts both conductive bands simultaneously, a circuit is completed and a sensor/wireless transmitter sends a signal to a wireless receiver to set off an alarm.

One of the problems associated with a diaper having electronic wires and the like incorporated in the structure of the diaper is the expense of fabricating the diaper. Another problem is one of waste in that after one use, the diaper is discarded along with the incorporated electron wires and any other electronics incorporated therein.

Accordingly, it is an object of the invention to provide an economically reusable diaper monitor.

It is another object of the invention to reduce the cost of monitoring for incontinence in seniors.

It is another object of the invention to provide a diaper monitor that can be readily incorporated into diapers of different manufacture at the time of use.

It is another object of the invention to provide a diaper monitor that can be readily removed from a wetted diaper for re-use in other diapers.

Briefly, the invention provides a diaper monitor for use in a diaper or like structure for the monitoring of a wetting of the diaper by a wearer.

The diaper monitor is constructed to be handled as a small unit that can be inserted into a diaper at the time of use of the diaper. In this respect, the diaper monitor may be stored separately from a diaper and slipped into the diaper at the time of use or the diaper monitor may be stored with the diaper and removed from the diaper after wetting of the diaper for another use.

In accordance with the invention, the diaper monitor is comprised of a sensor housing having a pair of parallel spaced apart conductive strips on a surface of the housing that are positioned to close a circuit upon wetting of the area between the strips. These strips may extend over one surface of the monitor or may be extended about one end of the monitor. In addition, the sensor has a wireless transmitter sealed within the housing that is responsive to completion of an electrical circuit between the conductive strips to emit a signal. In this respect, the transmitter is constructed as described in US Patent Application 2012/0206265, the disclosure of which is incorporated herein.

In addition, the diaper monitor has a clip mounted on the housing for movement between a locking position to secure the housing to a diaper and an open position to allow removal of the housing from a diaper.

The monitor may be constructed wherein the wireless transmitter includes an electrical circuit, a battery for energizing the circuit and a switch for selectively connecting the battery to the circuit.

In one embodiment, the clip may be used to close the switch of the wireless transmitter when in the closed position of the clip and to open the switch in the open position of the clip. For example, the housing may include a first pair of electrical contacts on an outside surface in electrical communication with the switch while the clip includes a second pair of electrical contacts for contacting the first pair of contacts when the clip moves into the locking position. In this case, the monitor may be stored for future use with the clip in the open position to avoid draining the battery thereby extending the life of the battery. Upon insertion in a diaper and closing the clip, the battery is activated and the transmitter is ready for use.

In another embodiment, the monitor is constructed to have the wireless transmitter activated at the time of use at a remote station. For example, the wireless transmitter may be activated by inserting the monitor into or passing the monitor across a scanning device, for example at a nurse's central station, that effects a closing of the switch. After activation, the monitor may be inserted in a diaper on a wearer. This remote station may also have a recording means for receiving the signal from the monitor indicative of a wetting of the inner layer of a diaper on a user.

One advantage of using a scanning device to activate the monitor, is that information may be recorded regarding the wearer with whom the monitor is to be associated, the time of day, the monitor, such as serial number, and the like. Subsequent scanning of the monitor may also provide information regarding the period of use of the monitor, and the like.

The diaper monitor is particularly adapted for use with a diaper having a liquid protective outer layer and at least one liquid absorbent inner layer. In accordance with the invention, the monitor is removably mounted in the diaper for sensing wetting of the inner layer. For example, where the diaper and monitor are provided separately, the outer layer of the diaper may be slit to form an opening through which the housing of the monitor may then be inserted. After insertion of the housing so that the conductive strips are in contact with the absorbent layer, the clip of the monitor is moved into the locking position to engage the outer layer of the diaper between the clip and housing thereby holding the monitor in place for use.

In order to secure the clip in the locking position, the clip includes a bar-like projection and the housing has a pair of spaced apart parallel rails for selectively receiving the bar-like projection and the outer layer of the diaper therebetween in snap-fit relation. The clip may also be formed as a wire clip that can be snap-fitted into a pair of rails.

The forward end of the sensor housing may have a wedge or tapered forward end or a rounded forward end to facilitate sliding through a slit in the outer layer of a diaper.

After a diaper has been wetted and is to be discarded from a wearer, the clip on the monitor is moved to the open position to allow removal of the housing from the diaper and the monitor is slipped out of the diaper. After cleaning and sterilizing, the monitor can be re-used in another like diaper.

These and other objects of the invention will become more apparent taken in conjunction with the accompanying drawings wherein:

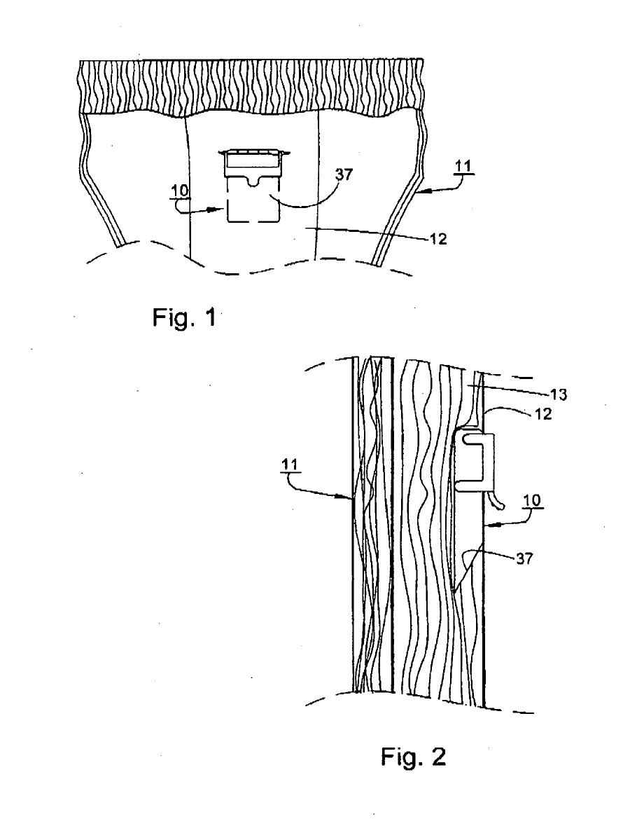

FIG. 1 illustrates a part perspective view of a diaper having a monitor in accordance with the invention;

FIG. 2 illustrates a view taken on line 2-2 of FIG. 1;

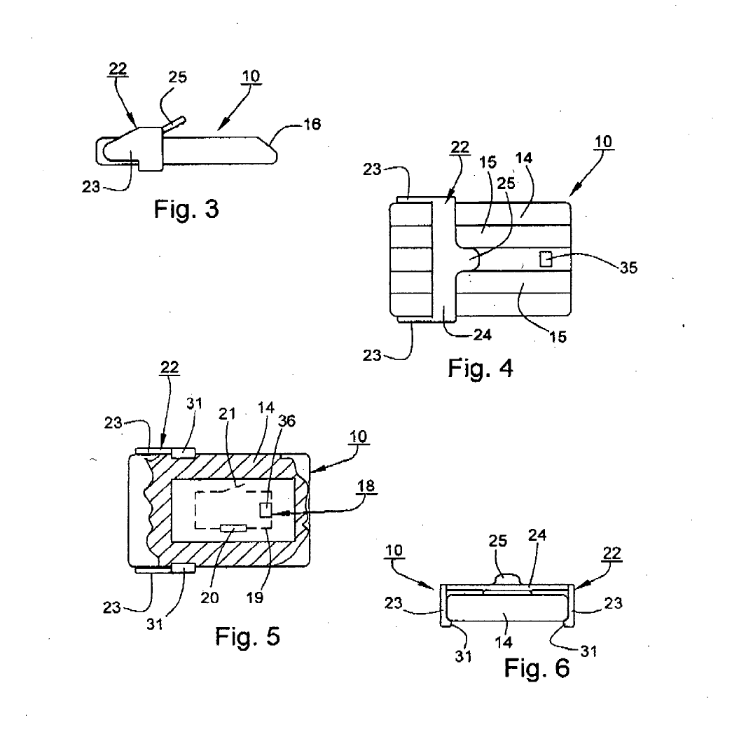

FIG. 3 illustrates a side view of the monitor of FIG. 1;

FIG. 4 illustrates a top view of the monitor of FIG. 1;

FIG. 5 illustrates a broken bottom view of the monitor of FIG. 1;

FIG. 6 illustrates a front view of the monitor of FIG. 1;

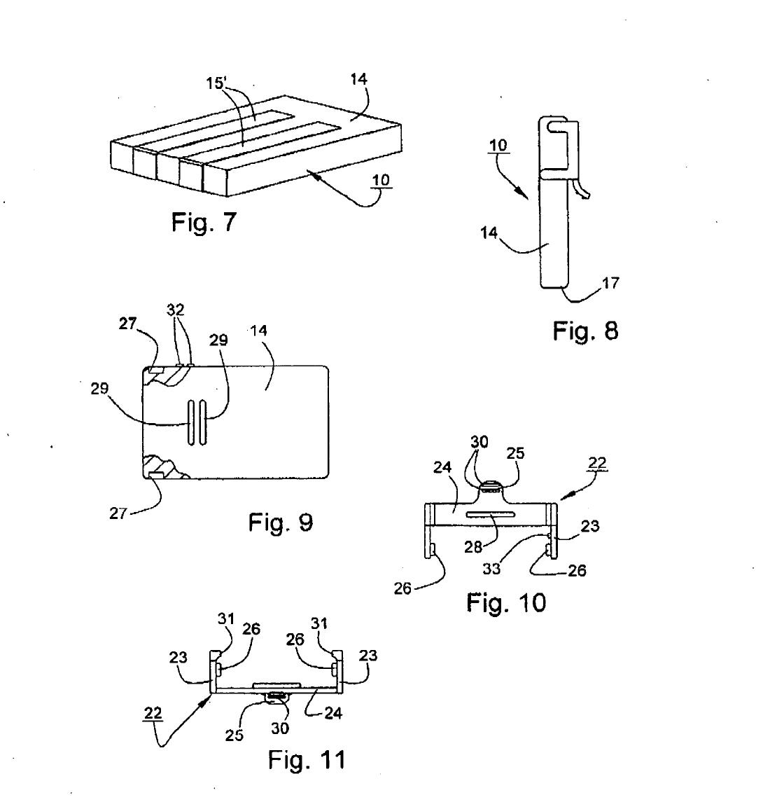

FIG. 7 illustrates a perspective view of a monitor with contact strips extending about one end;

FIG. 8 illustrates a side view of a monitor housing with a rounded forward end in accordance with the invention;

FIG. 9 illustrates a broken top view of a sensor housing having a pair of parallel rails to receive a bar-like projection of the, clip and electrical contacts on the sides;

FIG. 10 illustrates a bottom view of a clip having a bar-like projection in accordance with the invention;

FIG. 11 illustrates an end view of the clip of FIG. 10;

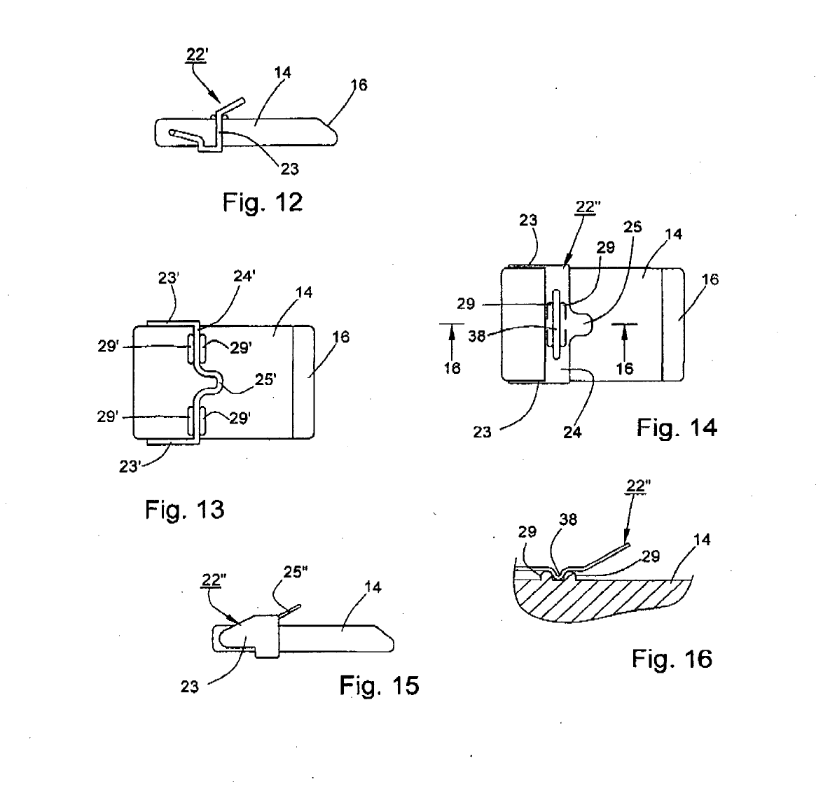

FIG. 12 illustrates a side view of a monitor housing with a modified clip in accordance with the invention;

FIG. 13 illustrates a top view of the monitor of FIG. 11;

FIG. 14 illustrates a top view of another modified clip in accordance with the invention;

FIG. 15 illustrates a side view of the monitor and clip of FIG. 14; and

FIG. 16 illustrates a view taken on line 16-16 of FIG. 14.

Referring to FIG. 1, the diaper monitor 10 is used with a diaper 11 or like incontinent pad. In this regard, the diaper 11 has a liquid protective outer layer 12 and at least one liquid absorbent inner layer 13. The diaper is of otherwise conventional structure.

The monitor 10 is removably mounted in the diaper 11 for sensing wetting of the inner layer 13.

Referring to FIGS. 3 to 6, the monitor 10 includes a sensor housing 14 having a pair of parallel spaced apart conductive strips 15 for contacting with the absorbent layer 13 of the diaper 11. As illustrated, the housing 14 is of flat rectangular shape with a tapered forward end 16 and is made of plastic or any other suitable material. Alternatively, as indicated in FIG. 8, wherein like reference characters indicate like parts as above, the housing 14 may have a rounded forward end 17.

As illustrated in FIG. 4, the conductive strips 15 extend over one surface of the housing 14 to be exposed to the absorbent inner layer 13 of the diaper 11. Alternatively, the conductive strips 15′ may extend around an end of the housing 14 as illustrated in FIG. 7 to the underside. In either case, the strips 15, 15′ may be applied in any suitable manner, for example, by being stamped in place, formed by a conductive paint, bonded in place or mounted as separate strips or bands on the housing 14.

Referring to FIG. 5, the monitor 10 includes a wireless transmitter 18 within the housing 14 that is responsive to completion of an electrical circuit between the conductive strips 15 to emit a signal therefrom. The wireless transmitter 18 includes an electrical circuit 19, a battery 20 for energizing the circuit 19 and a normally open switch 21 for selectively connecting the battery 20 to the circuit 19. In this respect, the wireless transmitter 18 is of the same type and construction as the transmitter in published US Patent Application 2012/0206265.

Referring to FIGS. 3 and 4, the monitor 10 includes a clip 22 that is mounted on the housing 14 for movement between a locking position, as shown, for engaging the outer layer 12 of the diaper 11 (see FIG. 1) and an open position (not shown) to allow removal of the monitor from the diaper 11. As illustrated, the clip 22 is pivotally mounted at one end of the housing 14 to pivot between the locking position and open position.

Referring to FIGS. 3, 4 and 6, the clip 22 is made of plastic and has a pair of legs 23 pivotally mounted on the housing 14, a crossbar 24 integral with and transverse to the pair of legs 23 and a projection 25 extending from the crossbar 24 for manual grasping thereof.

Referring to FIGS. 9 and 10 , each leg 23 of the clip 22 carries an integral boss 26 of circular shape at a free end for fitting into a circular recess 27 in a side of the housing 14 (see FIG. 9) in order to allow for pivoting of the clip 22 relative to the housing 14. The crossbar 24 of the clip 22 carries an integral bar-like projection 26 on an inside surface facing the housing 14 for reception between a pair of spaced apart parallel rails 29 on the housing 14 in snap-fit relation. The projection 25 of the clip 22 carries a pair of projecting ribs 30 that provide a roughened surface for manual gripping by a finger.

Referring to FIG. 11, each leg 23 of the clip 22 has an inwardly projecting foot 31 for engaging under the housing 14 (see FIGS. 3 and 5) when the clip 22 is in the locking position. In this regard, as the clip 22 moves from an open position to the locking position (FIG. 3), each leg 23 flexes outwardly as each foot 31 slides over a side of the housing 14 to snap into place under the housing 14.

The clip 22 is constructed to close the switch 21 in the transmitter 18 upon movement into the locking position and to open the switch 21 when moved to the open position. To this end, the housing 14 includes a pair of electrical contacts 32, for example, on an outside surface (see FIG. 9) that are in electrical communication with the switch 21 of the wireless transmitter and the clip 22, when made of plastic, includes an electrical contact 33 (see FIG. 10) for electrically connecting the contacts 32 on the housing 14 with the clip 22 in the locking position.

Alternatively, when the clip is made of electrically conductive material, the electrical contacts 32 may be placed on opposite sides of the housing 14 in order to be contacted by the legs 23 of the clip 22 in the closed position.

In another embodiment, the monitor 10 is constructed to have the wireless transmitter 18 activated at the time of use by inserting the monitor into or by passing the monitor 10 across a scanning device (not shown), for example at a remote station, such as a nurse's central station, that effects a closing of the switch 21. After scanning and activation, the monitor 10 may be inserted in the diaper 11 for use on a wearer. In this embodiment, the switch 21 is movable into a closed position thereof in response to an activation signal delivered remotely from the scanning device.

The remote station may also have a recording means for receiving and recording, for example, the serial number of the monitor 10, the time of activation of the monitor 10, the time of a signal from the monitor 10 indicative of a wetting of the inner layer of a diaper on a user. Where the signal from the monitor 10 is continuous during completion of the electrical circuit due to the diaper being wet, when the signal ceases in response to the diaper becoming dry, the recording means responds by recording the time at which the signal ceases.

Further, Referring to FIG. 4, the housing 14 of the monitor 10 may have a transparent section 35 while, as shown in FIG. 5, the wireless transmitter 18 includes a light bulb 36 for emitting a light, such as a green light, in response to the switch 21 connecting the battery 20 to the circuit for viewing through the transparent section 35 of the housing 14.

After manufacture, the monitor 10 may be stored for an extended period of time before being put into service.

In order to place the monitor 10 in service, where the monitor 10 has electrical contacts 32 as in FIG. 9, the monitor 10 is activated at the time of moving the clip 22 into the closed position upon being inserted into a diaper 11. In this case, a signal may be emitted from the wireless transmitter 18 to a remote process controller as described in US Patent Application 2012/0206265 to indicate that the monitor has been activated.

Where the monitor 10 is activated at a scanning device (not shown) with the light bulb 36 being illuminated, a user can readily determine that the monitor 10 is ready for use in a diaper by viewing the emitted green light through the transparent section 35 of the monitor 10.

Referring to FIG. 1, in order to use the monitor 10, the outer layer 12 of the diaper 11 is slit to form a pocket 37 (see FIG. 2) into which the monitor 10 may be manually inserted. Typically, the monitor 10 would be inserted with the clip 22 in an open position. After locating the housing 14 of the monitor 10 in the pocket 37, the clip 22 is pivoted manually into the closed position, as illustrated, thereby sandwiching the lip of the outer layer 12 about the pocket 23 between the housing 14 and the clip 22. At this time, the bar-like projection 28 on the crossbar 24 of the clip 22 and the parallel rails 29 on the housing 14 clamp the outer layer 12 of the diaper 11 therebetween while snap-fitting together.

In a case where the diaper 11 has a preformed pocket 37, the user need only slide the monitor 10 into the pocket 37 and clip the monitor in place.

Once in place, the monitor 10 is employed in a manner as described in published US Patent Application 2012/0206265 to monitor any wetting of the absorbent layer 13 of the diaper.

Upon removal of a diaper from a wearer, the clip 22 of the monitor 10 is moved to the open position to de-energize the electrical circuit 19 and the monitor 10 slid out of the pocket 37. The monitor 10 can then be cleaned and sterilized for another use in a diaper. Typically, the housing 14 of the monitor 10 is constructed in a seal tight manner to protect the interior surfaces and, particularly, to protect the wireless transmitter 18. Further, the housing 14 may be filled with silicone to encapsulate the contents of the housing 14.

When the clip 22 is moved to the open position, the wireless transmitter 18 may be programmed to emit a signal to the remote process controller to record the time of de-activation of the monitor 10.

Referring to FIGS. 12 and 13, wherein like characters indicate like parts as above, the clip 22′ may be made of a bent wire with a pair of straight sections forming a crossbar 24′ sized to be snap fitted between a pair of rails 29′ on the monitor housing 14 with the outer layer 12 of a diaper 11 therebetween. In addition, a projection 25′ is formed between the straight sections of the clip 22′ to project at an acute angle from the housing 14 for manual lifting away from the housing 14.

In this embodiment, the two ends of the wire clip 22′ are fitted into bores (not shown) in the sides of the monitor housing 14 so that the wire clip 22′ is pivotally mounted on the housing 14.

When the wire clip 22′ is moved into the closed position as indicated in FIGS. 12 and 13, the outer layer 12 of a diaper 11 (see FIG. 1) is gripped between the housing 14 and clip 22′ and, particularly, between the legs 23′ of the clip 22′ and the sides of the housing 14 as well as between the crossbar 24′ and the top surface of the housing 14.

Alternatively, as indicated in FIGS. 14 to 16, wherein like characters indicate like parts as above, the clip 22″ may be made of a stainless steel stamping or other suitable material. As above, this clip 22″ has a pair of legs 23 pivotally mounted on the housing 14, a crossbar 24 integral with and transverse to the pair of legs 23 and a projection 25 extending from the crossbar 24 for manual grasping thereof.

Referring to FIGS. 14 and 16, the crossbar 24 has a depressed portion 38 in a central region for snap-fitting between a pair of parallel rails 29 on the housing 14 with the outer layer 12 of a diaper 11 therebetween.

The invention thus provides a diaper monitor that need not be built into a diaper and one that can be stored separately from a diaper. As such, the invention provides an economically reusable diaper monitor.

The invention also provides a diaper monitor that allows for a reduction in the cost of monitoring for incontinence in seniors, that can be readily incorporated into diapers of different manufacture at the time of use and that can be readily removed from a wetted diaper and cleaned for re-use in other diapers.

The invention also allows the battery life of the monitor to be extended by opening the circuit to the battery when the clip is in an open position and closing the circuit to the battery only when the clip is moved into the closed position.

What is claimed is:

1. A diaper monitor comprising
a sensor housing having a pair of parallel spaced apart conductive strips on a surface thereof and a wireless transmitter sealed within said housing, said transmitter being responsive to completion of an electrical circuit between said conductive strips to emit a signal therefrom; and
a clip mounted on said housing for movement between a locking position to secure said housing to a diaper and an open position to allow removal of said housing from a diaper.

2. A diaper monitor as set forth in claim 1 wherein said conductive strips extend from said surface to around an end of said housing opposite said clip.

3. A diaper monitor as set forth in claim 1 wherein said housing is made of plastic.

4. A diaper monitor as set forth in claim 1 wherein said housing is of rectangular shape and has a tapered forward end.

5. A diaper monitor as set forth in claim 1 wherein said housing is of rectangular shape and has a rounded forward end.

6. A diaper monitor as set forth in claim 1 wherein said wireless transmitter includes an electrical circuit, a battery for energizing said circuit and a switch for selectively connecting said battery to said circuit.

7. A diaper monitor as set forth in claim 6 wherein said clip closes said switch in said locking position thereof.

8. A diaper monitor as set forth in claim 7 wherein said housing includes a pair of electrical contacts on an outside surface thereof in electrical communication with said switch and said clip includes an electrical contact for electrically connecting said pair of contacts with said clip in said locking position thereof.

9. A diaper monitor as set forth in claim 6 wherein said switch is movable into a closed position thereof in response to an activation signal from a remote location.

10. A diaper monitor as set forth in claim 6 wherein said switch is movable into a closed position thereof in response to an activation signal delivered thereto at a remote location.

11. A diaper monitor as set forth in claim 1 wherein said signal is continuous during completion of said electrical circuit

12. A diaper monitor as set forth in claim 1 wherein said clip has a pair of legs pivotally mounted on said sensor housing, a crossbar integral with and transverse to said pair of legs and a projection extending from said crossbar for manual grasping thereof.

13. A diaper monitor as set forth in claim 12 wherein said clip includes a bar-like projection on said crossbar and said sensor housing has a pair of spaced apart parallel rails for selectively receiving said bar-like projection therebetween in snap-fit relation.

14. A diaper monitor as set forth in claim 1 wherein said housing has a transparent section and said wireless transmitter includes an electrical circuit, a battery for energizing said circuit, a switch for selectively connecting said battery to said circuit and a light bulb for emitting a light in response to said switch connecting said battery to said circuit for viewing thereof through said transparent section of said housing.

15. A diaper monitor as set forth in claim 1 wherein said clip is made of stainless steel.

16. In combination,
a diaper having a liquid protective outer layer and at least one liquid absorbent inner layer; and
a monitor removably mounted in said diaper for sensing wetting of said inner layer, said monitor including a sensor housing having a pair of parallel spaced apart conductive strips in contact with said absorbent layer and a wireless transmitter within said housing and responsive to completion of an electrical circuit between said conductive strips to emit a signal therefrom, and a clip mounted on said housing for movement between a locking position engaging said outer layer and an open position to allow removal of said housing from said diaper.

17. The combination as set forth in claim 16 wherein said outer layer of said diaper is engaged between said sensor housing and said clip in said locking position of said clip.

18. The combination as set forth in claim 16 wherein said outer layer of said diaper forms a pocket receiving said diaper monitor.

19. The combination as set forth in claim 16 wherein said housing is of rectangular shape and has a tapered forward end disposed between said outer layer and said absorbent layer.

20. The combination as set forth in claim 16 wherein said wireless transmitter includes an electrical circuit, a battery for energizing said circuit and a switch for selectively connecting said battery to said circuit and said clip opens said switch in said open position thereof and closes said switch in said locking position thereof.

21. The combination as set forth in claim 20 wherein said housing includes a pair of electrical contacts on an outside surface thereof in electrical communication with said switch and said clip includes an electrical contact for electrically connecting said pair of contacts with said clip in said locking position thereof.

22. A diaper monitor as set forth in claim 16 wherein said clip has a pair of legs pivotally mounted on said sensor housing, a crossbar integral with and transverse to said pair of legs and a projection extending from said crossbar for manual grasping thereof.

23. The combination as set forth in claim 16 wherein said wireless transmitter includes an electrical circuit, a battery for energizing said circuit and a switch for selectively connecting said battery to said circuit.

24. The combination as set forth in claim 23 further comprising a remote station for receiving and activating switch to a closed position to connect said battery to said circuit

25. The combination as set forth in claim 24 wherein said remote station has recording means for receiving said signal from said monitor indicative of a wetting of said inner layer of said diaper.

26. The combination as set forth in claim 25 wherein said signal is continuous during completion of said electrical circuit and said recording means is responsive to a completion of said continuous signal to indicate drying of said inner layer of said diaper.
